(19) United States
(12) Patent Application Publication
(10) Pub. No.: US 2018/0340226 A1
(43) Pub. Date: Nov. 29, 2018

(54) SYSTEM AND METHOD OF DIAGNOSING ENDOTHELIAL DYSFUNCTION UTILIZING CIRCULATING MIRNAS AS BIOMARKERS

(71) Applicant: Serenium, Inc., Palo Alto, CA (US)

(72) Inventors: David Gozal, Chicago, IL (US); Leila Gozal, Chicago, IL (US)

(73) Assignee: Serenium, Inc., Palo Alto, CA (US)

(21) Appl. No.: 15/779,087

(22) PCT Filed: Aug. 22, 2016
(86) PCT No.: PCT/US2016/048065

§ 371 Date: May 25, 2018

Related U.S. Application Data
(60) Provisional application No. 62/207,828, filed on Aug. 20, 2015.

(51) Int. Cl. C12Q 1/6883

(52) U.S. Cl. CPC C12Q 1/6883; C12Q 2600/178; C12Q 2600/158

(57) ABSTRACT

It was determined that plasma-derived exosomes from either obese (OB) or obstructive sleep apnea (OSA) children with evidence of endothelial dysfunction (ED). Such ED exosomes lead to up-regulation of adhesion molecules in endothelial cells. Exosomal miRNA cargo differences underlie the mechanisms accounting for the presence of ED. Specifically, expression of miRNA-630 is reduced in circulating exosomes of either obese or OSA children with ED, and normalizes in OSA children with ED after treatment along with restoration of endothelial function. These findings elucidate a novel role of exosomal miRNA-630 as a putative key mediator of vascular function and a biomarker of cardiovascular disease (CVD) risk in children.

### PRIORITY

This application claims priority from U.S. Provisional Patent Application No. 62/207,828, filed Aug. 20, 2015, the disclosures of which are incorporated herein.

### FIELD OF THE INVENTION

The present invention relates generally to systems and methods for diagnosing endothelial dysfunction (ED) and, more particularly, to systems and methods for diagnosing ED utilizing circular miRNAs as biomarkers for the presence of ED.

### BACKGROUND AND METHODOLOGY OF THE INVENTION

Obese children are at increased risk for developing obstructive sleep apnea (OSA), and both of these conditions are associated with an increased risk for endothelial dysfunction (ED), an early risk factor for cardiovascular disease (CVD) in children. Although weight loss and treatment of OSA improve endothelial function, not every obese child or child with OSA will develop ED. Exosomes are nano-sized membrane vesicles frequently found in plasma, containing functional mRNA and miRNA that can be delivered to other cells, such as endothelial cells. According to the present inventive method, it was learned that differential miRNA within circulating exosomes may underlie the dichotomous vascular phenotypes in obese or OSA children.

Methodology for the present inventive method contemplates plasma exosomes from either obese children or non-obese OSA children, primarily derived from endothelial cell sources, recapitulated ED or its absence in human endothelial cells, and also in vivo when injected in mice. Furthermore, expression of miRNA-630 is reduced in circulating exosomes of children with ED, and normalizes after therapy along with restoration of endothelial function. Conversely, transfection of exosomes from subjects without ED with a miRNA-630 inhibitor induces the ED functional phenotype. Gene target discovery experiments further revealed that miRNA-630 regulates 416 gene targets in endothelial cells that include the Nrf2, kinase, and tight junction pathways. The present inventive method indicates a novel role of exosomal miRNA-630 as a putative key mediator of vascular function and CVD risk in children, and identifies putative therapeutic targets.

### SUMMARY OF THE INVENTION

Obesity is a frequent condition in children that carries a substantial risk for cardiovascular disease (CVD). Similarly, obstructive sleep apnea (OSA) is a highly prevalent pediatric disorder that is typically associated with a higher risk for cardiovascular morbidity, primarily presenting as increased systemic blood pressure deregulation that can lead to ventricular remodeling, as well as altered endothelial function, an early precursor of atherosclerosis. Furthermore, obese children are at significantly higher risk for OSA. However, not every child, even with severe OSA or obesity, displays abnormal endothelial function as measured by post-occlusive hyperemic responses, and the mechanisms underlying the determinants of such CVD susceptibility remain unclear. Thus, extraordinary efforts have been directed to determine the molecular and pathological characteristics of the diseased heart and vasculature, to develop novel diagnostic and therapeutic strategies.

Intercellular communication is an essential hallmark of multicellular organisms and can be mediated through direct cell-cell contact or transfer or secreted molecules. In response to physiological and/or pathological signals, all cells communicate with each other via secretion of a heterogeneous mixture of vesicles differing in size and composition. Exosomes, the most well-studied of these vesicles, are 30-100 nm vesicular structures which contains a wide variety of proteins, lipids, RNAs, non-transcribed RNAs, miRNAs and small RNAs. These diverse cargos allow exosomes to provide unique opportunities for biomarker discovery and development of non-invasive diagnostic when examined in biological fluids such as urine and blood plasma. Exosome content exchange represents a potential and intriguing pathway of intercellular communication by delivery of microRNAs, with the latter being implicated as reliable reporters of cardiovascular morbidity, as well as constituting potential therapeutic targets.

In the methodology of the present invention it was determined that plasma-derived exosomes from either obese (OB) or OSA children with evidence of endothelial dysfunction (ED) as indicated by abnormally delayed post-occlusive reperfusion kinetics exert differential effects on endothelial cell monolayer impedance and tight-junction function when compared to exosomes from either OB or OSA children with normal endothelial function (NEF). Such ED-exosomes lead to up-regulation of adhesion molecules in endothelial cells. Also, it was discovered that surgical adenotonsillectomy treatment of OSA in non-obese children with ED reverses exosome-induced changes. In addition, it was determined that exosomal miRNA cargo differences underlie the mechanisms accounting for the presence of ED. Specifically, expression of miRNA-630 is reduced in circulating exosomes of either obese or OSA children with ED, and normalizes in OSA children with ED after treatment along with restoration of endothelial function. Furthermore, transfection of exosomes from NEF subjects with a miRNA-630 inhibitor induces the ED functional phenotype, while transfection of exosomes from ED children with a miRNA-630 mimic abrogates the abnormal endothelial function. These experimental approaches further revealed that miRNA-630 is associated with a transcriptomic target signature in naive endothelial cells in culture. Taken together, these findings elucidate a novel role of exosomal miRNA-630 as a putative key mediator of vascular function and CVD risk in children, and identify putative therapeutic targets.

### DESCRIPTION OF THE DRAWINGS

FIG. 1 depicts a characterization of plasma-derived exosomes in children with and without endothelial dysfunction and exosomal characteristics using Bionalyzer, western blots and electron microscropy. Quality control assessment of total RNA was used for microarray analysis. Total RNA including miRNAs were isolated from plasma. Panel (A) shows a representative of the bio-analyzer profile for total RNA from plasma, and Panel (B) shows total RNA including plasma exosomal-miRNAs. RNA content was strikingly different, with the majority of exosomal RNA below 200 bases and with little or no bands of 28S and 18S ribosomal RNA, compared to cellular RNA. The fluorescence is depicted on the Y-axis and the time (s) on the X-axis. Panel (C) is a Western blot showing the absence of CD63 protein in plasma proteins, but the presence of the CD63 protein, which is commonly enriched in exosomes (Panel C). Electron micrographs of exosomes with the typical morphology and size (30-120 nm) are shown in (Panel D). Using flow-cytometric approaches exosomes from ED subjects (either obese or OSA) were primarily derived from endothelial cell (EC) or endothelial cell progenitors (ECP), when compared to corresponding obese or OSA children with normal endothelial function (NEF) (Panel E).

FIG. 2 depicts exosome-mediated in vitro effects on endothelial cell monolayer resistance and in vivo vascular function. Panel A: Representative image of plasma-derived immunofluorescently-labeled exosome after administration to primary human endothelial cells showing their incorporation into the cells. Below it, scattergram of Tmax, a measure of endothelial function in obese or OSA children, plotted against plasma-derived exosome effects on endothelial cell monolayer resistance (ECIS) at 24 hours. (R². 0.85, p<0.0001; n=41); Panel B: Illustrative ensemble averaged curves of ECIS changes over time after administration of exosomes from control children (CO), and obese children with (OBed) and without (OBnet) endothelial dysfunction (n=12/tracing); Panel C: Typical examples of post-occlusive reperfusion kinetics (5 min occlusion time) in a mouse injected once daily for 3 days with i.v. exosomes from a child with OSA and normal endothelial function (OSAnef) and in a mouse injected with exosomes from an age gender- and BMI z score-matched child with OSA with endothelial dysfunction (OSAed) illustrating the increased delay in time to peak reperfusion (Tmax) in the mouse injected with ED-derived exosomes; Panel D: Representative Tmax measurements in mice elicited by injection of exosomes from age-, gender-, BMI z core-matched OSAed and OSA nef children. Panel E: Tmax values in mice injected with exosome from control children (CO). OSAed. OSAnef, Obed, and OBnef (n=8/group; line indicate p<0.001). Panel F: Representative images of at least 6 separate experiments showing exosome-induced changes in expression of V-cadherin, ZO-1, ICAM-1, and VCAM1 in primary human endothelial cells after treatment with exosomes from obese children with (OBed) and without (OBnef) endothelial dysfunction, and from children with OSA and normal endothelial function (OSAnef) and age, gender, AHI and BMI-z score-matched children with OSA and endothelial dysfunction (OSAed). The scale bars for all the representative images are 25 μm.

FIG. 3 depicts effects of miRNA-630 mimic and inhibitor in ED and NEF-derived exosome functional changes in endothelial cell monolayer resistance and ZOI. Panel A: Left: Representative image of plasma-derived immunofluorescently-labeled exosomes after transfection with ref-fluorescence labeled miRNA-630 mimic and administration to primary human endothelial cells. Below the image, illustrative ensemble averaged curves of ECIS changes over time after administration of exosomes from 6 OSAed children treated with miRNA-630 mimic or scrambled mimic sequence against control empty exosomes. Middle: Summary of changes in normalized monolayer resistance measurements at 24 hours after treatment with control empty exosomes, exosomes from OSAed transfected with miRNA-630 mimic or scrambled mimic sequence, and exosomes from OSAnrf children treated with miRNA-630 inhibitor or scrambled sequence (n=8-12/experimental group). Right: Summary of changes in normalized monolayer resistance measurements at 24 hours after treatment with control empty exosomes, exosomes from OBed transfected with miRNA-630 mimic or scrambled mimic sequence, and exosomes from OBnef children treated with miRNA-630 inhibitor or scrambled sequence (n=8-12/experimental group). Panel B: Representative images of at least 6 separate experiments showing exosome-induced changes in expression of ZO-1 in primary human endothelial cells after treatment with exosome from obese children with (OBed) and without (OBnef) endothelial dysfunction, and from, children with OSA and normal endothelial function (OSAnef) and age, gender. AHI and BMI-z score-matched children with OSA and endothelial dysfunction (OSAed). Exosome from ED subjects were transfected with miRNA-630 mimic or scrambled mimic while exosomes from NEF subjects were transfected with miRNA-630 inhibitor or scrambled sequence as control.

FIG. 4 depicts miRNA-630 gene targets in human endothelial cells. Left heatmap depicts differentially expressed genes as determined via experiments comparing the effects of exosomes from 7 ED children treated with either miRNA-630 mimic or control. Right heatmap shows differentially expressed genes as determined by experiments comparing the effects of exosomes of 8 NEF subjects treated with miRNA-630 inhibitor and 7 NEF subjects treated with scrambled control. These experiments revealed a total of 416 gene targets in endothelial cells, corresponding to 10 major functional pathways, of which Nrf2 and tight junction pathways are shown for illustration purposes. Several of the putative miRNA-630 gene targets identified in the mRNA arrays were subsequently verified using qRT-PCR strategies.

### DETAILED DESCRIPTION OF THE INVENTION

While the invention may be susceptible to embodiment in different forms, there is shown in the drawings, and herein will be described in detail, specific embodiments with the understanding that the present disclosure is to be considered an exemplification of the principles of the invention, and is not intended to limit the invention to that as illustrated and described herein.

While preferred embodiments of the present invention are shown and described, it is envisioned that those skilled in the art may devise various modifications of the present invention without departing from its spirit and scope. Information about the experimentation that led to the development of the present invention follows.

### Subjects

The study that lead to the development of the inventive methodology was approved by the human subject committee of each of the participating centers (University of Chicago IRB Protocol# #09-115-B and Comite Etico de Investigacion Clinica del Area de Salud de Burgos y Soria protocol #603), and informed consent was obtained from the legal caregiver of each participant. Consecutive healthy obese or non-obese pre-pubertal children (ages 4-12 years) from the community, and children being evaluated for snoring who were polysomnographically diagnosed with OSA were invited to participate. All participants underwent baseline anthropometric and blood pressure assessments, as well as overnight polysomnography, which were interpreted using standard approaches. Measurements of endothelial function followed by a fasting blood draw were performed in the morning. The time to maximal regional blood flow after occlusion release (Tmax) is representative of the post-occlusion hyperemic response, an index of nitric oxide (NO)-dependent endothelial function. A Tmax>45 sec was considered as indicative of abnormal endothelial function. A nearly identical test was performed in 8-12 week-old C57b16 mice except that occlusion time was set at 5 minutes.

Distinctly different groups of children were identified in the study that led to the present inventive methodology: Controls: healthy non-snoring children with normal polysornnographic test, BMI z score <1.34, and normal Tmax; Obese children (OB), i.e., BMI z score 1.65 but normal polysomnographic test with normal Tmax (OBnef) or abnormal Tmax (OBed); snoring children with abnormal polysomnographic findings confirming the presence of OSA, BMI z score 1.65, and either normal Tmax(OSAnef) or abnormal Tmax(OSAed).

Exclusion Criteria

Children found to be hypertensive or using anti-hypertensive drug therapies were excluded (n=14). Furthermore, children with diabetes (fasting serum glucose 120 mg/dL; n=17), with a craniotfacial, neuromuscular or defined genetic syndrome, and children on chronic anti-inflammatory therapy (n=11), or with any known acute or chronic illness were also excluded. In addition, children placed on sympathomimetic agents such as psychostimulants were not tested (n=22).

Blood Tests

Fasting blood samples were drawn by venipuncture in the morning immediately after endothelial function testing, and serum lipid concentrations were examined using standard laboratory techniques.

Exosome Isolation, Labeling and Characterization

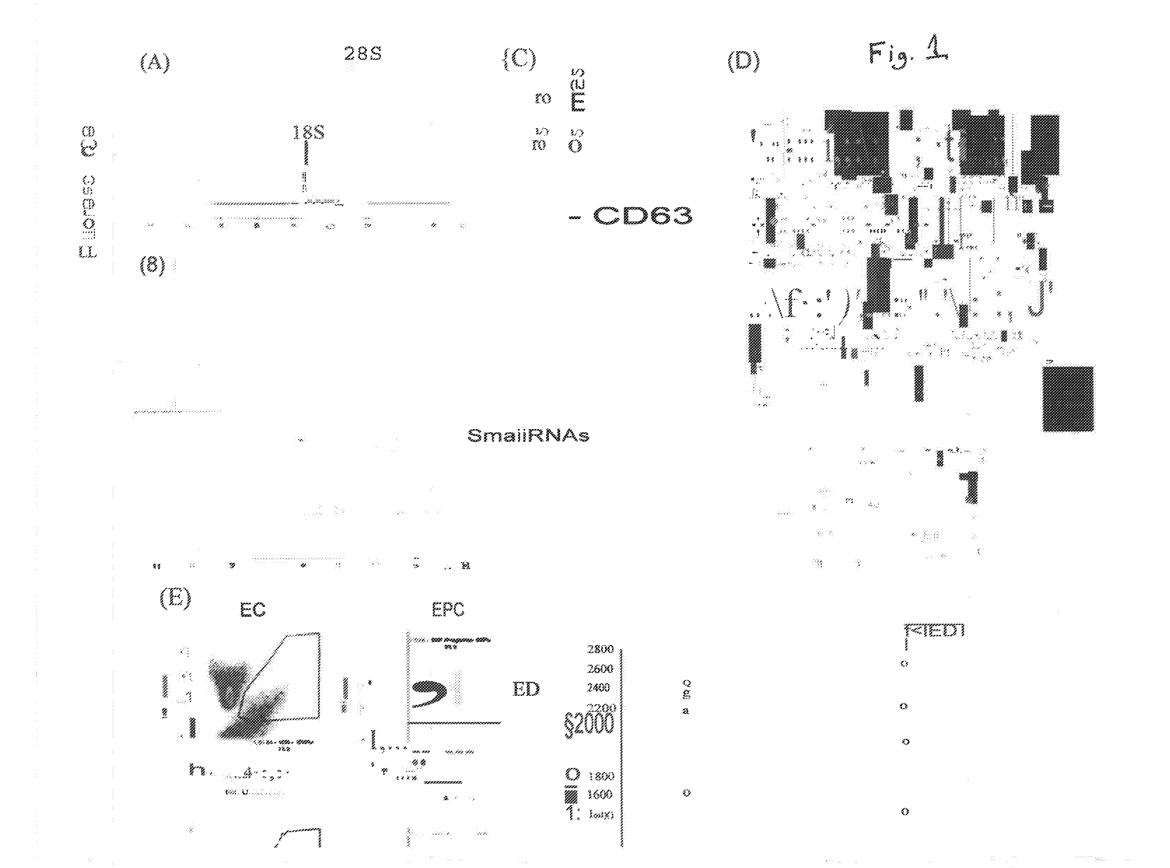

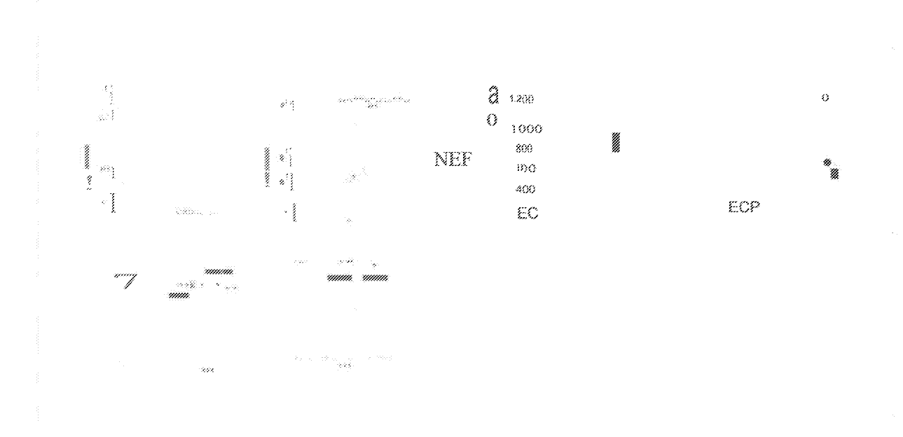

Exosome were isolated from plasma using standard approaches, were fluorescently labeled using Exo-Glow kit (cat# EXORIOOA-1; System Biosciences, Inc., Mountain View, Calif.), and delivered to endothelial cells in vitro or in vivo to mice. Similarly, cell source and size of exosomes were determined using FACS and electron microscope approaches as shown and described in FIG. 1.

Primary Endothelial Cell Culture

Human microvascular endothelial cells were purchased from Lonza (catalog #:CC-2543; Allendale, N.J.) and grown in Dulbecco's modified Eagle's medium supplemented with 4.5 g/L glucose, 3.7 g/L sodium bicarbonate, 4 mmol/L glutamine, 10% fetal bovine serum, 100 U/mL penicillin, and 100 mg/mL streptomycin, pH 7.4, and incubated at 37° C. and 5% CO2 in cell culture incubator. For continuous spas aging, the cells were trypsinized and centrifuged at 150×g for 7 min, diluted, and re-plated at appropriate densities. All cells were used before passage.

Electric Cell-Substrate Impedance Sensing (ECIS) and Immunofluorescence

Endothelial cells were grown to confluence into ECIS arrays as a single confluent monolayer, ECIS monitors the impedance of small 250-micrometer diameter electrodes used as substrates for cell growth, and as cells grow on the electrode they constrict electrical current flow altering the impedance. Exosomes were added in duplicate wells and placed into the ECIS instrument (Applied Biophysics Inc. Troy, N.Y.) for continuous monitoring up to 24 hrs. The baseline was established using culture medium (300 μl/well⁻¹) alone and compared with values obtained using electrodes covered with a monolayer of cells in 500 μL medium. For immunofluorescence staining, confluent endothelial cell monolayers were grown on 12 cover slips, after which isolated exosomes from subjects were added individually to cover slips for 24 hrs. Cells were fixed, permeabilized, and followed by overnight incubation at 4° C. with ZO-1, 1:400; and VE-cadherin, 1:400 (Life Technologies, Grand Island, N.Y., USA), ICAM-1 (1:250), and VCAM1, 1:100; Santa Cruz Biotechnology, Inc., Dallas, Tex.). Alexa 488 or Alexa-594 were used as secondary antibodies (1:400, 2 mg/ml; Life technologies, Grand Island, N.Y.) and nuclear staining with DAPI (4′,6-Diamidino-2-Phenylindole, Dihydrochloride); 1:1000; Life Technologies, Grand Island, N.Y.) were performed. Images were captured with a Leica SPS Tandem Scanner Spectral2-photon confocal microscope (Leica Microsystems, Inc., Buffalo Grove, Ill.) with a 63^{x} oil-immersion lens.

Exosome Cellular Uptake

To assess the cellular uptake of exosomes in a subset of experiments (n=6-8), exosomes were stained with ExoGreen stain, which is based on carboxyfluorescein succinimidyl diacetate ester chemistry (SBI, Mountain View, Calif.), and were imaged with a Leica SPS Tandem Scanner Spectral 2-photon confocal microscope (Leica Microsystems, Inc., Buffalo Grove, Ill.) with a 63^{x} oil-immersion lens.

miRNA Isolation and Microarrays

Total RNA including miRNA was isolated with exosomes using miRNeasy Mini Kit-column-based system following the manufacturer's instructions (Qiagen, Tumberry Lane, Valencia, Calif., USA, and profiled on the Agilent human miRNA Microarray (Agilent Technologies), consisting of 60-mer DNA probes synthesized in situ that represent 2006 human miRNA. After hybridization and washing, the arrays were scanned with an Agilent microarray scanner using high dynamic range settings, and results were extracted using Agilent Feature Extraction software (v11.0). A feature was listed as detectable if >50% of the signals in at least one of the two groups were above the detection limit (‘well above background’ flag=1) and a probe was considered to be detectable if >50% of the replicated features were detectable.

Gene Ontology (GO) and Functional Annotation

Analysis of GO annotation was performed by applying DAVID (the Database for Annotation. Visualization and Integrated Discovery (DAVID 6.7) functional annotation tool. This DAVID software (http://david.abcc.nciferf.govi) is able to identify the most relevant (over-represented) biological terms associated with a given gene list. The DAVID functional annotation cluster tool groups genes based on their associated GO annotations, and the related terms are clustered into groups with emichment scores calculated from their EASE Score, the modified Fisher Exact P-value. Moreover, the web server hosts an updated version of the KEGG database providing a relevant search module based on KEGG pathway descriptions.

miRNAs Mimics and Inhibitors In Vitro and In Vivo

Exosome transfections were performed using the Exo-Fect™ Exosome Transfection Reagent as described by the manufacturer's protocol (cat# EXFT20A-1; System Biosciences, Inc. SBI, Mountain View, Calif., USA). Briefly, 50 μl of purified exosome (100 μg) was used in each reaction and the following reagents were added: 10 μl Exo-Feet solution, 20 μl (20 μmol mimic miRNA, or inhibitor miRNA), 70 μl steril1×PBS. The mixtures were incubated at 37° C. in a shaker for 10 minutes and then 300 μl of the Exoquick-TC were added to stop the reactions. The samples were centrifuged at 13,000 rpm for 3 minutes. The transfected exosome pellets were resuspended in 300 μl 1×PBS, and used in the ECIS system for the miRNAs mimics and inhibitors of interest (75 μl were added to approximately 5×10⁵ cells/well in a 12-well culture plate grown in exosome-depleted FBS, and reaching equivalent exosome concentrations across conditions. miRNAs mimics and inhibitors were purchased from Life Technologies (Grand Island, N.Y., USA). And were labeled with ExoRed, which is based on Acridine Orange chemistry (SBI, Mountain View, Calif.). The specifically desired increase or decrease in miRNA-630 exosomal content by the miRNA-630 mimic or inhibitor, respectively, was verified using qRT-PCR.

### Data Analysis

Results are presented as means±SD, unless stated otherwise. All numerical data were subjected to statistical analysis using independent Student tests or analysis of variance followed by post-hoc tests (Tukey) as appropriate. Chi square analysis was performed on categorical data concerning demographic characteristics of the various groups. Pearson correlation testing was conducted to establish association between several study parameters, including ECIS-derived normalized resistance changes and Tmaxin endothelial function tests. Finally, canonical correlation analyses were performed to explore the relationships between sets of variables. Statistical analyses were performed using SPSS software (version 21.0; SPPS Inc., Chicago, Ill.). For all comparisons, a 2-tailed p<0.05 was considered to define statistical significance.

### Results

### Subject Characteristics

A total of 128 children completed the study. Their demographic and polysomnographic characteristics are provided in Table 1. There were no significant differences in age, gender, ethnicity among the 2 OSA sub-groups, but the OSAed children showed slightly higher BMI z scores than OSAnef or CO children (Table 1). No differences in BMI z scores were present in OBed and OBnef There were no significant differences in the severity of OSA, as indicated by either the obstructive AHI or the nadir SpO₂ levels, in systolic and diastolic blood pressures, or in lipid profiles between OSAed and OSAnef children, but these measures were significantly different from CO children (Table 1). As anticipated from the definition of ED, both OBed and COSAed groups had markedly prolonged Tₘₐₓ (56.8÷8.6 sec. and 54.7÷8.5 sec), compared to either OBnefF (33.2±7.3 sec; p<0.01), OSAnef (31.6±6.2 sec; p<0.01) or CO (29.9±5.1 sec; p<0.01). A subset of 16 OSAed children was treated by surgical removal of tonsils and adenoids and underwent a second assessment as per protocol within 4-8 months after surgery. These children showed normalization of their respiratory disturbance during sleep (AHI decreased from 15.7±4.7/hr sleep to 0.7±0.4 sleep, p<0.001), and in all children but one, normalization of endothelial function occurred (Tₘₐₓ decreased from 57.7±8.5 sec to 37.8±7.3 sec. p<0.001).

TABLE 1
General characteristics of obese children and children with OSA with and without
endothelial dysfunction and healthy controls.

| | OBNEF | OBED | OSANEF | OSAED | Control |
|---|---|---|---|---|---|
| | (n = 23) | (n = 20) | (n = 34) | (n = 25) | (n = 26) |
| Age (yers) | 7.6 ± 2.6 | 7.7 ± 2.8 | 7.3 ± 2.4 | 7.4 ± 2.3′ | 7.3 ± 2.2 |
| Gender (male, %) | 56.5 | 60 | 50 | 56 | 58 |
| Ethnicity (Caucasian, n) | 6 | 6 | 11 | 9 | 8 |
| BMI Z score | 1.74 ± 0.28 | 1.76 ± 0.31 | 1.17 ± 0.22 | 1.42 ± 0.27* | 1.08 ± 0.21* |
| Systolic blood pressure (mmHg) | 113.2 ± 8.6 | 114.0 ± 9.1 | 108.2 ± 8.4 | 112.8 ± 9.2 | 102.3 ± 8.4* |
| Diastolic blood pressure | 68.7 ± 7.9 | 68.9 ± 8.3 | 65.8 ± 7.2 | 67.8 ± 7.8 | 60.9 ± 6.7** |
| (mmHg) | | | | | |
| Obstructive AT:ll (events/hour) | 0.9 ± 0.4 | 0.8 ± 0.6 | 12.8 ± 8.4 | 13.1 ± 9.4 | 0.3 ± 0.3** |
| Sp02 Nadn · (%) | 93.2 ± 3.3 | 93.9 ± 4.1 | 75.9 ± 10.9 | 73.0 ± 11.9 | 92.1 ± 2.9** |
| Total Cholesterol (mg/dl) | 171.6 ± 37.3 | 173.4 ± 40.0 | 174.9 ± 36.1 | 178.3 ± 37.8 | 159.4 ± 19.3** |
| HDL cholesterol (mg/dl) | 48.9 ± 8.7 | 44.8 ± 9.2 | 44.7 ± 8.5 | 46.7 ± 8.8 | 59.3 ± 7.8** |
| LDL cholesterol (mg/dl) | 119.3 ± 19.9 | 121.8 ± 23.6 | 117.2 ± 24.5 | 120.5 ± 24.7 | 95.7 ± 20.3** |
| Triglycerides (mg/dl) | 107.2 ± 32.1 | 110.3 ± 33.9 | 105.7 ± 35.5 | 107.8 ± 36.7 | 93.7 ± 28.3* |
| Time to maximal hyperemic | 33.2 ± 7.3 | 56.8 ± 8.6 | 31.6 ± 6.2 | 54.7 ± 8.5** | 29.9 ± 5.1 |
| responses (Tₘₐₓ; sec) | | | | | |

*p < 0.05;
**p < 0.01

### Exosomal Cell Sources

FIG. 1 illustrates exosome characterization procedures. The overall concentrations of plasma-isolated exosomes among children with ED and NEF were similar. However, exosomes from ED children were increasingly derived from either endothelial cells or endothelial progenytor cells, when compared to those from matched NEF children with no significant differences for monocytes, T-cell lymphocytes, or neutrophils. The concentration of exosomes from platelet sources was increased only in children with OSA and ED, but not among obese children with ED without OSA. Indeed, OSA children had overall increased platelet derived exosomes (852±122/10,000 counts in OSAed, 367±89/10,000 counts in OSAnef, 228±78/10,000 counts in either OBed or OBnef; and 217±81/10,000 counts in controls; p<0.01 OSAed vs. all other groups).

### Exosomal Effects on Endothelial Cell Monolayers

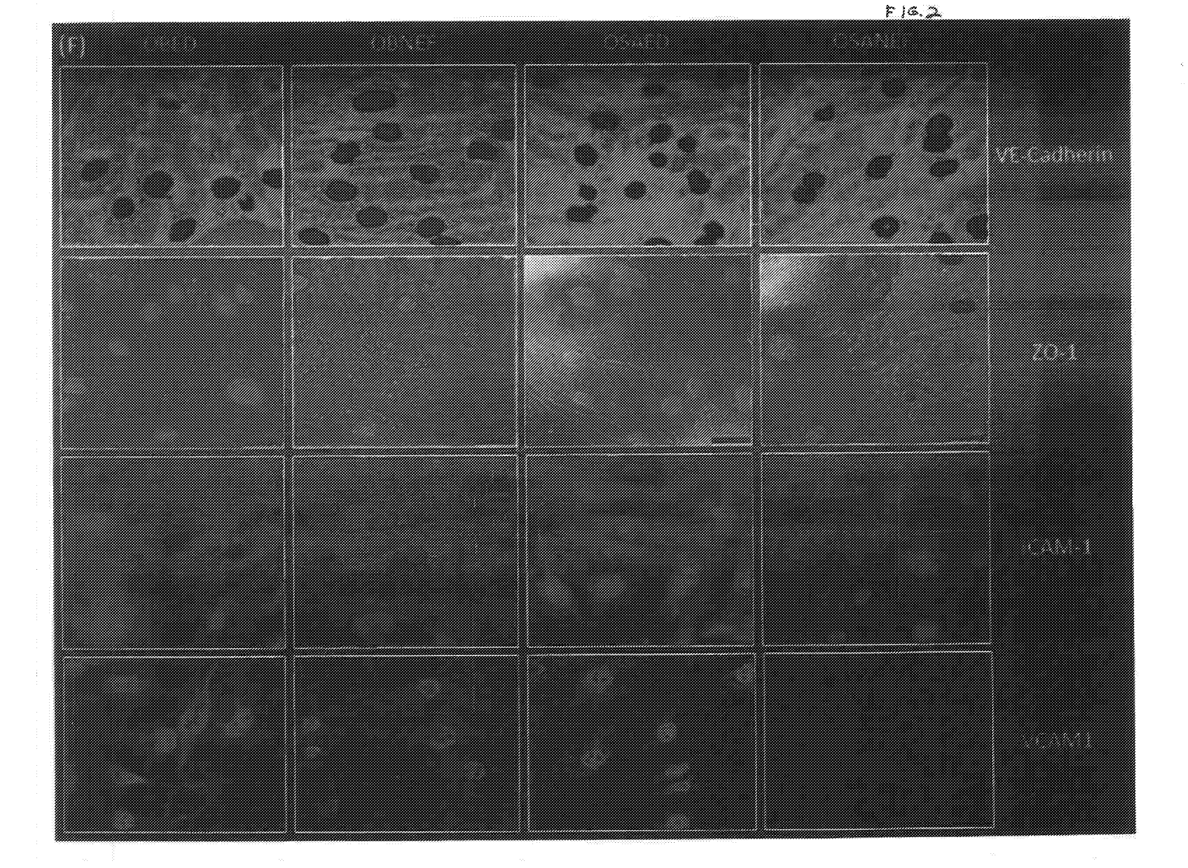

Human primary commercially available endothelial cells were cultured in a single monolayer and exposed to exosomes from NEF and ED children corresponding to both obese and OSA groups. ECIS normalized resistance values were continuously monitored, and revealed substantial decrements in monolayer resistance among exosomes from LEI) children, which were absent or markedly attenuated in NEF children (FIG. 2). Notably, ECIS values at either 6 hours (data not shown) or 24 hours were strongly associated with corresponding Tmax values (FIG. 2). Furthermore, ED-derived exosomes induced disruption of the endothelial cell membrane as illustrated by discontinuity of V-cadherin, altered topographic distribution of the tight junction protein ZO-1, and increased expression of adhesion molecules ICAM-1 and VCAM-1 (FIG. 2). Similarly, ED exosomes from either OSA or OB children, but not NEF exosomes (from either OB, OSA, or control children), induced significant reductions in expression of endothelial nitric oxide synthase (eNOS) mRNA, when compared to exosomes derived from healthy control subjects (ED: 57.6±8.9% vs. NEF: 2.9±5.2%; p<0.001). When plasma-derived exosomes isolated from children with ED (either OB or OSA) or NEF (OB, OSA, and controls) were i.v. injected into mice, and vascular function was assessed at 24 and 48 hours after injection, significant prolongations in Tmax occurred after administration of ED-derived exosomes (both obese and OSA), but were absent after treatment with NEF-derived exosomes when compared to control exosomes (FIG. 2). Adenotonsillectomy (Tx) treatment of OSA in the children with ED not only normalized their Tmax, but also led to normalization of plasma exosome-induced endothelial cell monolayer resistance changes (Pre-Tx: −37.8±7.4% vs Post-Tx: −4.7±4.6%; n=15; p<0.001).

Exosomal miRNA Cargo

Differences in miRNA exosomal content between NEF and ED children were initially explored using miRNA arrays, and revealed a restricted cluster of 5 miRNAs that were consistently altered across the two groups independent of obese or OSA status, with abundance of 4 miRNAs being reduced in ED (miRNA-16-5: 3.18 fold, p<0.0001; miRNA-451a: 3.74 fold; p<0.0001; miRNA-5100: 1.65 fold; p<0.01; and miRNA-630; 4.11 fold; p<0.0001) and increased abundance being found for 1 miRNA (miRNA-4665-3p; 2.35 fold; p<0.01). These findings were subsequently validated and confirmed in all groups (n=20/group) using qPCR. Furthermore, marked increases in exosomal miRNA-630 and miRNA-16-5p back to levels in healthy control children occurred in those children with OSA and ED after treatment (miRNA-630: Pre-Tx: 4.76±0.97 vs. Post-Tx 0.19±0.32, n=15; p<0.001; miRNA-16.5p: PeTx: −2.87±0.45 vs. Post-Tx: −0.87±0.65, p<0.01).

MiRNA-630 and Endothelial Function

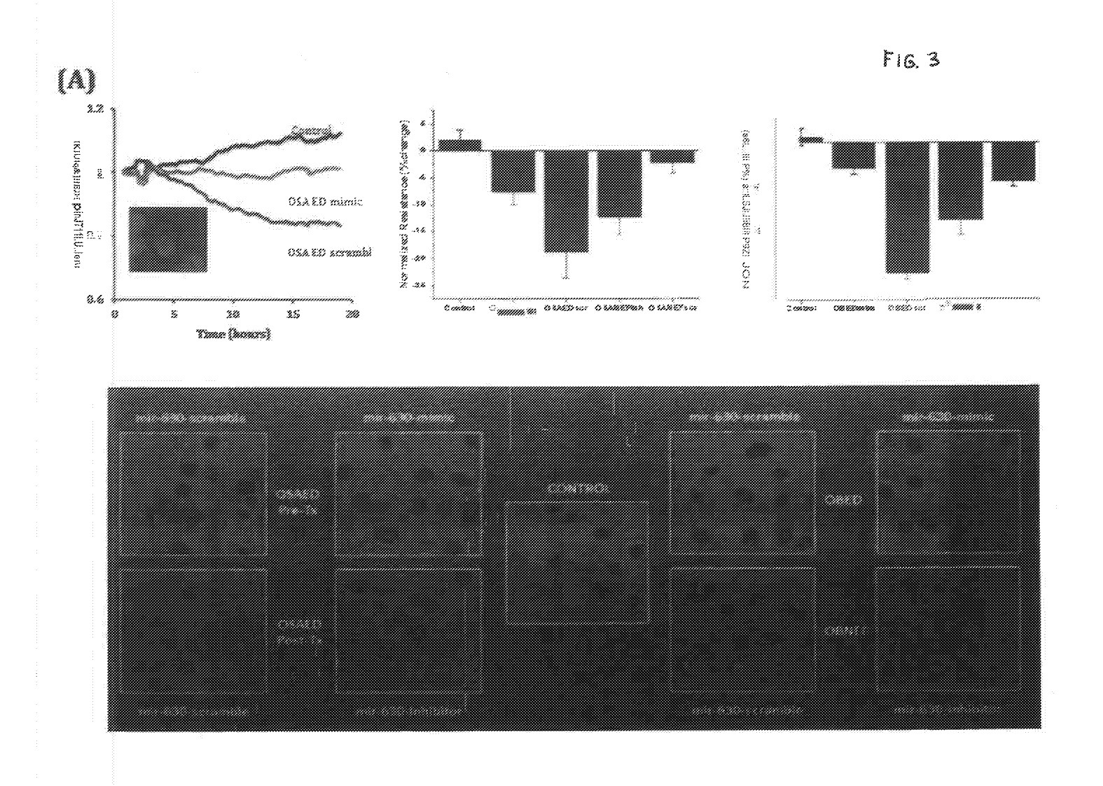

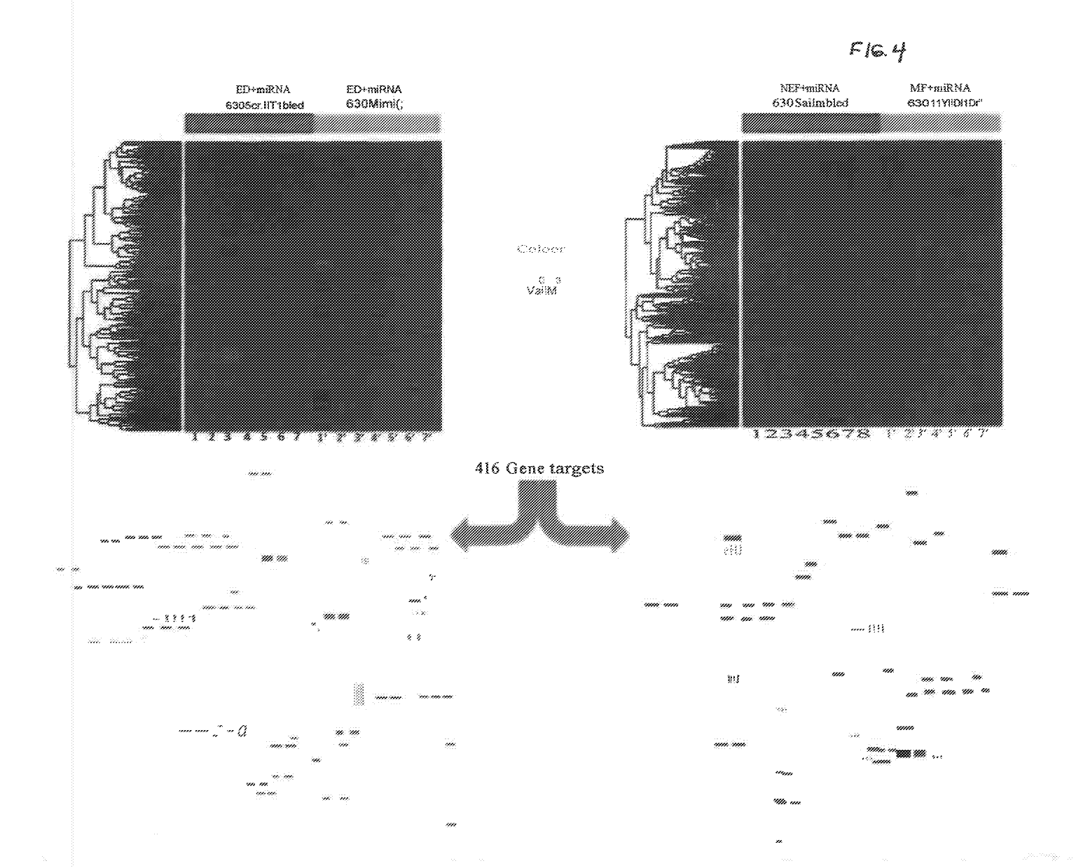

To explore the putative function of miRNA cargo in the context of pediatric ED, we selected miRNA-630 as the initial candidate based on the largest fold differences in expression between ED and NEF obese and OSA children, as well as the largest responses in OSAed children after Tx. To this effect, specific miRNA-630 mimic and inhibitor were transfected into plasma-derived exosomes from ED and NEF, respectively (the control treatment, scrambled sequences of the mimic and inhibitor were used). Mimic-induced restoration of miRNA-630 content in ED-derived exosomes significantly ameliorated the adverse ED exosome-induced effects on ECIS measures of resistance across the endothelial cell monolayer and ZO-1 tight junction immunofluorescence in endothelial cells for both obese or OSA children with ED (FIG. 3), and also restored eNOS mRNA expression using control exosomes as reference (0.56±0.17 in scrambled miRNA treated endothelial cells vs. 0.94±0.29 in mimic miRNA treated endothelial cells; n=4; p<0.05). Conversely, transfection of NEF-derived exosomes with a miRNA-630 inhibitor was associated with disruption of the monolayer resistance and altered ZO-1 endothelial cell distribution (FIG. 3), as well as reductions in eNOS expression in endothelial cells (0.47±0.22 following treatment with miRNA-630 inhibitor vs. 1.04±0.33 after scrambled miRNA; n=5; p<0.02).

MiRNA-630 Gene Targets in Endothelial Cells

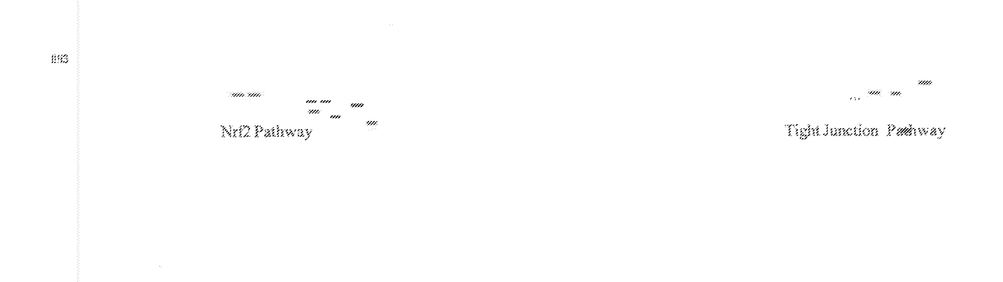

A schematic of the experimental subtraction-based approach for delineation of the miRNA-630 gene targets in human endothelial cells is shown in FIG. 4. Using whole genome transcriptomic analyses, a total of 416 gene targets were identified for miRNA-630 in endothelial cells (FIG. 3), and correspond to 10 major canonical pathways (Table 2). Notable among these pathways are for example NRF2-mediated oxidative stress responses, AMP kinase, and tight junction signaling pathways (FIG. 4).

TABLE 2
List of the top 10 canonical miRNA-630 gene target
pathways in endothelial cells as derived from 416 gene targets.

| | Ingenuity Canonical Pathways | p-value |
|---|---|---|
| 1 | NRF2-mediated Oxidative Stress Response | 8.19E−05 |
| 2 | Regulation of eIF4 and p70S6K Signaling | 1.08E−04 |
| 3 | Protein Ubiquitination Pathway | 2.46E−04 |
| 4 | Chronic Myeloid Leukemia Signaling | 3.33E−04 |
| 5 | Cell Cycle Regulation by BTG Family Proteins | J3.90E−04 |
| 6 | AMPK Signaling | 2.26E−04 |
| 7 | Role of BRCA1 in DNA Damage Response | 4.59E−04 |
| 8 | Hereditary Breast Cancer Signaling | 4.29E−03 |
| 9 | DNA Double-Strand Break Repair | 5.73E−03 |
| | by Homologous Recombination | |
| 10 | Tight Junction Signaling | 1.24E−03 |

Discussion and Conclusions

This study shows that plasma exosomes from children with ED, either obese or suffering from OSA, induce marked in vitro and in vivo functional and structural alternations in endothelium that are mediated by altered exosomal miRNA cargo that primarily originates from endothelial cells and endothelial cell progenitors. Among the differentially expressed exosomal miRNAs, reduced miRNA-630 expression emerged as a significant effector of ED, and restoration of miRNA-630 exosomal levels appeared to correct the endothelial cell perturbations in ED-derived exosomes, while inhibition of miRNA-630 in NEF-derived exosomes recapitulated the perturbations elicited by ED exosomal responses in endothelial cells. Taken together, we identify a microvesicle-based miRNA-mediated mechanism that is selectively altered in children who are at increased CVD risk.

Post-occlusive hyperemic tests provide a robust and reliable non-invasive method for assessment of endothelial function, while enabling accurate reporting on the bioavailability of nitric oxide from eNOS sources in the circulation. Furthermore, incremental evidence attests to the vascular remodeling that progressively occurs in the context of ED since early childhood to increase CVD risk later in life. In the current study, we identified two a priori different conditions which are both associated with increased risk for vascular dysfunction, namely obesity and OSA. The degree of eNOS expression changes in naYve cultured endothelial cells treated with exosomes from ED or NEF subjects markedly different, independently from whether the exosomes originated from obese children or from non-obese children with OSA. Furthermore, changes in monolayer resistance in ECIS experiments were closely associated with Tₘₐₓ, suggesting that the changes in eNOS expression and in monolayer resistance following exosome exposure provide accurate biological correlates of endothelial function in vivo. Furthermore, intravenous administration of equivalent doses of exosomes from the various subject groups to otherwise healthy young mice generated markedly discrepant changes in post-occlusive hyperemic responses in these animals, that paralleled the original findings in children. These experiments provided conclusive evidence that the biological properties of circulating exosomes in children are sufficient to induce the vascular phenotype, and therefore prompted exploration of the exosomal cargo to identify potential candidates that may mediate the vascular deficits observed in a subset of children with either obesity, OSA, or both. However, due to the restricted amount of plasma available for any given child, we cannot deduce from current studies as to the specific contributions of exosomes from different cells sources to the in vitro or in vivo endothelial functional abnormalities.

Several reports have shown that miRNAs are actively secreted in exosomes from different cell types, highlighting their potential to serve as paracrine intercellular signaling molecules. The selection of these exosomal miRNA as the major discovery effort was not random, but rather reflected previous work implicating exosomal miRNAs not only in angiogenesis and other vascular phenomena, but also suggesting the possibility that exosomes may provide therapeutic opportunities. Furthermore, we recently addressed the potential role of plasma miRNAs as candidate biomarkers of vascular dysfunction, and preliminary findings would support the driving assumption that a cluster of circulating miRNAs may reliably discern between ED and NEF children.

Among the 5 differentially miRNAs identified through microarray approaches, a search of the literature revealed scarce information about the majority of these miRNAs with regard to possible vascular targets and tissue expression. In this context, miRNA-630 was selected for further examination based on the prominent reduction in expression among ED children, the marked increases in miRNA-630 in exosomes of children after treatment of OSA, and its previous association with angiogenesis and apoptotic processes in the context of cancer. Of note, miRNA 16-5p has been implicated in cardiac cell functions in the context of heart failure, and identified in pericardial fluid. Based on the current consensus that miRNAs bind to their mRNA targets and induce their down-regulation, decreases in miRNA levels could then possibly be associated with increased expression of specific target genes and proteins. The experiments in which a miRNA-630 mimic and inhibitor were incorporated into exosomes enabled specific assessments of the functional role of miRNA-630 in endothelial cells, and confirmed the mechanistic role of this exosomal miRNA in the vasculature. Furthermore, transcriptomic strategies identified 416 gene targets for this miRNA in endothelium that clearly encompass well established pathways in vascular homeostasis, such as Nrf2, AMP kinase pathways, and regulation of intercellular tight junctions 42-44.

In summary, we have shown that a dichotomous vascular outcome, such as the presence or absence of endothelial dysfunction in pediatric OSA or in obese children without OSA, as indicted by post-occlusive hyperemic responses, can be recapitulated by in vitro or in vivo effects of plasma exosomes. Such exosome-induced alterations non naive endothelial cells orurine vascular responses can be explained, at least in party, by changes in miRNA-630 expression and its downstream coordinated effects on approximately 416 gene targets. Improved understanding of the mechanisms responsible for the differential individual vascular susceptibility to OSA or obesity in children should allow for improved delineation of disease phenotypes, and for formulation of more individualized therapeutic strategies in those children at risk for future CVD.

We claim:

1. A system and method of diagnosing endothelial dysfunction as described herein, in any embodiment and any configuration.

2. A system and method of diagnosing and identifying pediatric patients at risk for developing cardiovascular disease comprising diagnosing endothelial dysfunction using a selective signature of miRNA from plasma or from plasma derived exosomes.
